(19) United States
(12) Patent Application Publication
(10) Pub. No.: US 2021/0205589 A1
(43) Pub. Date: Jul. 8, 2021

(54) CATHETER AND CLICK CONNECTOR

(71) Applicant: CRM Medical Devices, Inc., Mountain View, CA (US)

(72) Inventors: Meilin Dong, Mountain View, CA (US); Christopher Stephen Elliott, Mountain View, CA (US); Rustin Massoudi, Mountain View, CA (US)

(73) Assignee: CRM Medical Devices, Inc., Mountain View, CA (US)

(21) Appl. No.: 17/141,887

(22) Filed: Jan. 5, 2021

Related U.S. Application Data
(60) Provisional application No. 62/957,933, filed on Jan. 7, 2020.

(51) Int. Cl. A61M 25/10; A61M 1/00

(52) U.S. Cl. CPC A61M 25/1025; A61M 25/10186; A61M 2210/1085; A61M 1/0019; A61M 25/10183

(57) ABSTRACT

In one embodiment, the present application discloses, in part, a balloon catheter assembly comprising:
a) a balloon catheter comprising a proximal end, a distal end; and
b) a connecting line comprising a proximal end and a distal end, wherein the proximal end comprises a first terminal magnetic connector for forming a water tight connection with the magnetic click connector on the port panel.

### CROSS-REFERENCE TO RELATED APPLICATION

This application claims the priority under 35 USC 119(e) of U.S. Application No. 62/957,933, filed Jan. 7, 2020, entitled “Balloon Catheter and Click Connector”, which is incorporated into this application by reference.

### FIELD OF THE INVENTION

In one embodiment, the present application relates to a balloon catheter, catheter valves, a fixator, and a click connector for use with the catheter. In particular, the application discloses catheters and devices for communicating with the bladder or other body lumens or organs.

Lifelong bladder dysfunction is a common problem for those with neurologic disease. Amongst those unable to volitionally void due to neurogenic bladder (NGB) dysfunction are substantial numbers with spinal cord injury (SCI), spina bifida, and multiple sclerosis (MS). Compared to the general population, those with neurological disease face many challenges, which include bladder management difficulties that significantly reduce quality of life and increase medical complication rates. The impact of bladder-care difficulties is illustrated by the fact that the majority of SCI patients feel that improving their bladder condition is more important than regaining the ability to walk.

For NGB patients who are unable to volitionally void, clean intermittent catheterization (CIC), the act of passing a catheter through the urethra into the bladder every 4-6 hours, is a commonly used approach to bladder management. Unfortunately, more than half of those unable to volitionally void due to NGB have challenges that limit or preclude the use of CIC. The two largest groups of patients who experience difficulty independently performing CIC are those with limited upper extremity (UE) motor function and women (due to difficulties in accessing their urethra). As a result, CIC is not a realistic independent bladder management option for many. These difficulties are mirrored in experience suggesting that “CIC dropout” is common and occurs in about 50% of individuals who are started on intermittent catheterization for NGB dysfunction.

Patients with poor UE motor function face multiple challenges performing CIC including: the act of independently opening a catheter from a sealed package; removing ones' clothing to access the urethra; guiding the catheter through the urethra into the bladder; holding a urine receptacle during bladder drainage; urine and material disposal after bladder drainage; and re-dressing. Consequently, CIC can frequently only be performed with the aid of a caregiver. Even when adequate upper extremity motor function is preserved, most women attempting to perform CIC also struggle to perform the task independently. Women experience difficulty obtaining urethral access due to the urethras internalized location at the vaginal orifice which is often further exacerbated by poor lower body strength, lower body spasticity and positioning difficulties. As a result, most women are unable to access their urethra in a wheelchair and instead must transfer out of their wheelchair to catheterize. The extra steps necessary can be extremely time consuming and it is estimated that women in this circumstance can spend up to 2 hours per day simply attending to their bladder needs with significant decreases in quality of life.

The required planning, time spent catheterizing and overall inconvenience of performing CIC in the groups above, results in more than half of NGB patients ultimately choosing alternative bladder management options in the long-term. These alternatives include involuntary leakage into diapers, indwelling catheters (urethral or suprapubic) with passive urinary drainage into a receptacle, or major reconstructive surgery, all of which are associated with increased long-term morbidity compared to CIC.

Leakage into a diaper can lead to losses in renal function, and in some cases progress to renal failure. In addition, the chronic dampness from having the skin constantly exposed to urine further increases the risk of decubitus ulcers with erosion into soft tissue and/or bone. Chronic leakage also results in the stigma of always smelling of urine and is a significant adverse barrier to socialization and sexual activity.

Indwelling catheters, which drain via passive means, are complicated by a substantial risk of tube encrustation and blockage (leading to autonomic dysreflexia, febrile UTI and bladder stone formation (˜70% lifetime risk)). Indwelling catheters also carry the stigma of having a tube in the body attached to a bag of urine at all times, and in the case of urethral catheters (which are either in the penis or directly adjacent to the vaginal opening) interfere with sexual activity. In addition, long-term urethral catheterization often results in urethral erosion with genital disfigurement and in severe cases, uncontrollable urinary incontinence.

Reconstructive surgery to create a catheterizable channel from the bladder to the abdominal wall can increase the chance of successful CIC compared to using the urethra. A catheterizable channel is intended to remove urethral accessibility barriers, and decrease the amount of UE motor dexterity needed to catheterize. Unfortunately, continent catheterizable channels are complex surgeries with substantial recovery times and only offered by specialized physician providers. In addition, significant long-term morbidity accompanies catheterizable channel surgery, with 20% requiring revision for channel scarring or leakage and up to 10% abandoning its use despite the time and effort put into its creation.

Regrettably, since the introduction of CIC in 1972, the available options for those unable to perform CIC have not appreciably changed. This lack of innovation to improve bladder management choices continues to be a glaring omission and there is a critical need for innovation. Accordingly, there is a need for devices and methods that allow bladder drainage for those with neurogenic voiding dysfunction, specifically those who are unable to perform CIC due to UE motor or other anatomic limitations. The present inventors discovered that such an invention should address the key considerations and complications associated with commonly used CIC alternatives including:
Placement via a non-urethral approach to eliminate urethral injury and barriers to sexual activity;
Minimization of device encrustation compared to other indwelling tubes due to active drainage of urine from the bladder as opposed to passive drainage;
Safe device placement, preferably in the outpatient setting;
Elimination or avoidance of the long-term catheterizable channel reconstruction problems (stenosis, leakage, irreversibility);
Enabling use in persons with poor UE motor function and those that have difficulty accessing their urethra;
Providing easy connection and disconnection that can be performed with even with poor UE dexterity;
Avoidance of leakage including in low pressure, low viscosity environments, e.g., leakage of urine from the bladder;
Improving cosmesis (low profile with no urinary drainage bag attached to the patient), e.g., provide a low profile relative to a patient's skin or body such that the devices is inconspicuous;
Minimizing maintenance by requiring infrequent device changes; and
Addressing the needs of surgeons and other clinical users responsible for initial placement.

### DESCRIPTION OF THE RELATED ART

Balloon Catheters

A “balloon catheter” is a catheter having a balloon near and surrounding one end (the “distal end”) thereof, a tubular body comprising two lumens: a first lumen, referred to in this application as the “principal lumen”, through which fluids can flow; and a second lumen, referred to in this application as the “inflation lumen”, through which the balloon can be inflated, and at the other end (the “proximal end”) of the body, ports for connecting to the principal and inflation lumens. The inflated balloon retains the distal end of the catheter within a body cavity or organ, and fluids may be introduced into or drained from that cavity or organ through the principal lumen. The wall defining the inflation lumen typically lies completely inside the outer cross-section of the wall defining the principal lumen, so that the outer surface of the wall defining the principal lumen forms the outer surface of the catheter over most of the length of the body (i.e., the length excluding the distal and proximal ends).

A common example of the balloon catheter is the Foley catheter used to drain urine from the bladder. The catheter is inserted balloon-end-first through the urethra into the bladder, and the balloon is inflated with sterile water or the like to retain the balloon end of the catheter in the bladder. The same catheter can also be used as a suprapubic urinary catheter, where the catheter, instead of being inserted into the bladder through the urethra, is inserted into the bladder though a stoma (or tract), an incision through the skin of the lower abdomen and into the bladder. Other examples of balloon catheters are nutritional catheters (feeding tubes) used for parenteral nutrition and commonly referred to as a gastrostomy tube (“G-tube”) or jejunostomy tube (“J-tube”), when these catheters use a balloon for retention. These catheters are also placed with the balloon end within the stomach or jejunum, respectively, through a stoma, here an incision through the skin of the abdomen and into the stomach or jejunum, and the balloon inflated to retain that end of the catheter within the stomach or jejunum. While the Foley (urethral) catheter is typically retained in place only by the balloon inflated within the bladder, though U.S. Pat. No. 5,624,395 shows such a catheter with an external “retention collar” for use by female patients, suprapubic urinary catheters and nutritional catheters are typically also retained externally by some form of fixator adjacent the skin that ensures that the catheter does not move axially through the stoma in either direction: the inflated balloon and the external fixator define a fixed distance between the inner wall of the body organ and the skin.

Catheters with different design and function are known in the art. For example, U.S. Pat. No. 10,046,147 discloses, in part, a device for forming and/or maintaining a percutaneous access pathway into a body of a patient, comprising: an access pathway catheter configured to connect an internal portion of a body of a patient to an external environment; an access pathway port configured to maintain a barrier between the internal portion of the body and the external environment when in a closed position; an attachment device connectable to the access pathway port and configured to selectively cause the access pathway port to open by opening one or more doors in the access pathway; and an internally sterile attachment device sheath. U.S. Pat. No. 5,865,816 discloses, in part, a corporeal access tube assembly comprising a) a tube segment of predetermined length having a body; b) said body including a wall having an inner surface and an outer surface; c) said inner surface defining a passage extending from one end of said tube segment to the other end of said tube segment; d) said body wall containing a coil spring; e) a retention balloon fastened to and encircling said wall; f) a lumen in said body extending between said surfaces from a location in said tube segment. U.S. Pat. No. 9,149,415 discloses in part, an inflatable retention system for an enteral feeding tube having a base deployed outside the human body and an indwelling retainer which is deployed within a lumen of the body by insertion through a stoma from outside the body. U.S. Pat. No. 5,624,395 discloses in part, a urinary catheter for use by a person having a urethra with an orifice, said person further having an area of tissue surrounding said orifice of said urethra, said urinary catheter comprising: a body member, a retaining member; and an antiseptic substance. U.S. Pat. No. 8,633,268 discloses in part, indwelling urinary drainage catheters comprising a hypoallergenic or non-allergenic, synthetic polyisoprene based formulation.

A drawback to the use of balloon catheters when used as suprapubic urinary catheters or as feeding tubes is that for different uses and for different persons in whom the catheters are used, the desired distance between the balloon and the skin may differ substantially. This requires the physicians and institutions using such catheters to maintain a supply of catheters of different lengths.

In one aspect, it would be desirable to develop a balloon catheter that is capable of being emplaced within a body cavity or organ through a stoma (or tract), the balloon inflated to retain the catheter internally, an external fixator placed around the catheter at the skin surface to both retain the catheter against axial movement and to occlude the inflation lumen, and the catheter then be capable of being cut at or near that fixator, so that only one length of catheter or one form fitting catheter will be needed, with the length of the catheter for any particular patient being adjusted at the time of placement.

In another aspect, it would be desirable to provide a catheter incorporating an easily attachable and easily detachable connector to enable drainage through the catheter. In yet another aspect, it would be desirable to provide a connector that creates a tight seal when attached but remains easily attachable and detachable and further where a tight seal is maintained when detached.

The disclosures of the documents referred to in this application are incorporated into this application by reference.

### SUMMARY OF THE INVENTION

In one embodiment of the present application, there is provided a balloon catheter comprising a proximal end, a distal end, wherein the balloon catheter further comprises:
a catheter wall defining a catheter lumen;
an inflatable balloon comprising a proximal end and a distal end, where the inflatable balloon is mounted at the distal end of the catheter and the catheter further comprises a lumen that extends from the balloon to an inflation valve and inflation port;
a base located at the proximal end of the catheter, wherein the base comprises a top end and a bottom end, wherein the base comprises an opening to the catheter lumen, and the base further comprising:
   i) a port panel comprising a top end and a bottom end, and a port extending therethrough, wherein the top end of the port panel comprises a magnetic click connector for connecting with a connecting line comprising a corresponding magnetic click connector, and wherein the bottom end of the port panel is attached to the top end of the base; and
   ii) an inflation port for inflating the balloon, where the inflation port is mounted on or incorporated into the base and the inflation port further comprises an inflation valve. The inflation valve may be used for retaining fluid within the inflated balloon.

In a particular aspect, the base and the port panel together are a single component (or are of unitary construction), or may be made of the same material, such as a plastic. In one variation, the port panel may be made of a different material, such as a soft or hard plastic, from the material of the base, such as a different hard or soft plastic.

In another embodiment, the application discloses a magnetic click connector assembly for connecting a first medical device with a second medical device, a medical device with a connecting line, or a first connecting line with a second connecting line to provide a liquid tight or leak proof connection, the magnetic click connector assembly comprising:
a) a first magnetic click connector comprising a base further comprising at least one magnetic member; and
b) a second magnetic click connector comprising a base further comprising at least one magnetic member for magnetically coupled with the magnetic member of the first magnetic click connector to form a water tight connection;
   wherein the first magnetic click connector is coupled to a medical device or a connecting line and the second magnetic click connector is coupled to a medical device or a connecting line; and wherein each of the medical device and connecting line comprises a lumen therethrough for the passage of fluid.

In one aspect of the magnetic click connector assembly, the magnetic member comprises of a structure or shape selected from the group consisting of one or more pellets or beads, one or more rods, one or more circular rings, one or more semi-circular rings, or a combination thereof. In another aspect of the magnetic click connector assembly, the magnetic member of the first magnetic click connector comprises of a structure or shape that is the same or different from the second magnetic click connector. In another aspect of the above magnetic click connector, the magnetic member(s) are mounted on the base and are incorporated into the surface of the bases such that the magnetic member(s) may be fully encapsulated. As used herein, the term “mounted” means that the magnetic member(s) may be a part of the base or port panel, such as on the top part of the base, and may be incorporated into or on the base, and that the port panel may be connected to or otherwise made of seamless or unitary construction with the base. In one variation, the above catheter is equipped with a balloon as a retention element or device, alternatively or in addition, the retention element may comprise a malecot element or a mushroom element (i.e., malecot catheter) or J catheter to hold the catheter in place.

As used herein, the clause “a magnetic click connector for connecting with a connecting line comprising a corresponding magnetic click connector” and related clauses, mean that there is generally provided a first magnetic click connector and a second, configurationally similar or different (i.e., “corresponding”) magnetic click connector, as disclosed herein, for temporarily attaching two elements of a drainage device where fluid can flow between the two elements through a water tight seal and further where such a water tight seal may be enabled or improved by magnetic attraction of the two elements or components thereof.

In another aspect of the balloon catheter, the magnetic click connector comprises at least one magnetic member mounted on the base.

In another embodiment, there is provided a balloon catheter assembly comprising:

a) a balloon catheter comprising a proximal end, a distal end, wherein the balloon catheter further comprises:
a catheter wall defining a catheter lumen;
an inflatable balloon comprising a proximal end and a distal end, where the inflatable balloon is mounted at the distal end of the catheter and the catheter further comprises a lumen that extends from the balloon to an inflation valve and inflation port;
a base located at the proximal end of the catheter, wherein the base comprises a top end and a bottom end, wherein the base comprises an opening to the catheter lumen, and the base further comprising:
   i) a port panel comprising a top end and a bottom end, and a port extending therethrough, wherein the top end of the port panel comprises a magnetic click connector for connecting with a connecting line comprising a corresponding magnetic click connector, and wherein the bottom end of the port panel is attached to the top end of the base; and
   ii) an inflation port for inflating the balloon, where the inflation port is mounted on the base and the inflation port further comprises an inflation valve, where the inflation valve is in fluid communication with the balloon using an inflation lumen;

and

b) a connecting line comprising a proximal end and a distal end, wherein the proximal end comprises a first terminal magnetic connector for forming a water tight connection with the magnetic click connector on the port panel.

In one variation, the balloon catheter is a low profile balloon catheter. In another variation, the balloon catheter is an AMT Mini One® Balloon Button or a Kimberly-Clark Mic-Key® Balloon Button equipped with the above described base and magnetic click connector. The low profile catheter as disclosed herein do not have excess catheter tubing or catheter line beyond the skin level, other than the base of the catheter, also as disclosed herein. In another variation, the base of the catheter may be projected or directed off at a non-traditional angle, such as perpendicular from the patient's abdomen, or projected at a 15 degree, 20 degree, 25 degree, 45 degree, 60 degree, 75 degree or about 80 degree from the patient's abdomen, with respect to the catheter tubing. Such projected base at an angle that is non-perpendicular to the catheter tube allows a change of the catheter configuration to fit or accommodate body habitus; or where the tunneling that creates a need for a non-straight path to the bladder.

In one variation, the inflatable balloon may have an undetermined fill volume, or a predetermined fill volume. An inflatable balloon with an undetermined fill volume, for example, made from conventional elastic balloons that may be made from soft elastic silicone, for example, that are typically used with enteral feeding catheters. In one variation, the balloon catheter assembly may be urinary catheter or an enteral feeding catheter. Example of such similar basic structure and features for the balloon catheter include commercially available catheter devices include the AMT Mini One® Balloon Button and the Kimberly-Clark Mic-Key® Balloon Button, and further include the elements and features as disclosed herein.

In another aspect of the above balloon catheter assembly, the magnetic click connector of the top end of the port panel comprises one or more magnetic members mounted on the port panel for securely engaging or connecting the port panel and base to the connecting line.

The tightness of the water tight connection of the magnetic click connector comprising the magnetic members, as disclosed herein, for ease of connection and ease of disconnection may be controlled by the number, structure, shape and configuration of the magnetic members mounted on each magnetic click connector. In one aspect, each of the corresponding magnetic members of the magnetic click connector may be configured as one or more pellets or beads, such as 2, 3, 4, 5 or more pellets or beads that may be rectangular or round in shape; or each of the corresponding the magnetic members may comprise of one or more rods, such as 2, 3, 4, 5 or more rods, one or more semi circles, such as 2, 3 or 4 semi circles or a single circular ring, or other geometric configurations; or a combination thereof. In one variation, the pellets, beads, rods, semi-circle or circular rings, or combinations thereof, maybe configured as two or more concentric ring configuration to provide a stronger seal when connected together. In another variation, the complete top surface of the base comprising the magnetic member is a single magnetic member. As disclosed herein, one the magnetic members on a magnetic click connector and the corresponding magnetic click connector to be connected may be of similar or different structure and/or configuration. For example, the magnetic member on a first magnetic click connector may be a circular disk or ring, while the magnetic members on the second magnetic click connector may be a series of rods, discs, or pellets.

As disclosed herein the simplicity and ease of the self-locating and self-mating connectors and connecting mechanism to provide a leak tight connection provide a great advantage to users and patients with limited hand coordination.

In another aspect of the above balloon catheter assembly, the first terminal magnetic connector of the connecting line comprises a protrusion for engaging with a valve, such as a one-way valve, in the port to form a water tight connection.

In one variation, the valve is a one-way valve or a continence valve. In another variation the connection between the first terminal magnetic click connector and the magnetic click connector port panel further comprises an O-ring for forming a water tight seal. In one variation, the O-ring may be mounted inside, i.e., toward the inside lumen, of the magnetic members or outside of the magnetic members toward the outer wall; or both inside and outside for a tighter seal. As provided herein, the magnetic click connector would not allow the leakage of fluids through the lumen until the 2 magnetic click connectors are connected, thus mechanically opening the catheter's continence valve. In one variation, the balloon catheter assembly is made of glow in the dark material for ease of use at night or in limited light source.

In another aspect of the above balloon catheter assembly, the assembly further comprises a reversible locking mechanism or locking device for securing the two magnetic click connectors when connected, where the locking mechanism is selected from the group consisting of a twist lock, a locking ring, a locking sleeve, a male lock lever, cam lock coupling with or without arms and pins, a bayonet type twist locking mechanism for quick and easy connect and disconnect. In one variation, the assembly comprises a reversible locking mechanism or locking device for securing the two connectors that maybe non-magnetic connectors for other applications, such as for overnight use.

In another aspect of the balloon catheter assembly, the base further comprises a one-way valve in the port panel. In one aspect, the one-way valve prevents backflow and leakage of fluids from the line.

In one variation, the twist lock is a twist locking ring or twist collar or a quick clamp. The twist lock may be an adapter that, upon the connection of the magnetic click connector, the adaptor may be turned or rotated a half turn or a ¾ turn to lock the adaptor and connector in place. Such reversible locking mechanism may be a looping fastener, a ring or latch to further secure the magnetic connection. Such a fastener may be employed for example, when a patient goes to sleep to minimize the risk of unintentionally dislodging the connection during sleep. In one aspect, the reversible locking mechanisms of the magnetic click connector allows ease of use for patients with limited manual dexterity. In another aspect of the balloon catheter assembly, the base further comprises a plug, optionally tethered to the base.

In one variation, the plug or safety plug is used for plugging or capping the port when the port is not being used. In another variation, the plug is a continence plug. In another variation, the balloon catheter comprises at least one of an anti-reflux valve and a one-way valve.

In another aspect of the above balloon catheter assembly, the assembly further comprises an external pump for pumping fluids through the connecting line, or a bulb suction device for suctioning fluids.

In one variation, the connecting line is a drain line or a drain tube. In one variation, a mechanical bulb may be used to suction the fluid from the catheter and connecting line for discharge or removal of the fluid.

In one embodiment where the catheter is a urinary catheter, such as a urinary balloon catheter, the catheter may be employed to drain urine from a patient's bladder using standard methods known in the art or as disclosed herein. In another aspect of the above balloon catheter assembly, the assembly further comprises a drainage device selected from a drainage bag or a leg bag. In one variation, the leg bag comprises a strap for fastening of the leg bag on a patient's thigh or leg. As provided herein, the drainage device provides continuous gravity drainage. In another variation, the drainage device allows drainage while a patient is at rest, such as during a long sleeping period or for overnight rest. As disclosed herein, the leg bag may be employed during the day. In one variation, the drainage device comprises a capped drainage line or a drain spout that, upon opening via a cap or a twist closing mechanism, allows the collected urine to drain and be disposed, such as by draining into the toilet. In another variation, the drainage bag may be a disposable bag that may be detached or decoupled from the connecting or drainage line and be disposed. In another variation, the distal end of the connecting line further comprises a second terminal magnetic connector for forming a connection with a drainage bag or an outlet line for disposing fluids.

In another embodiment, there is provided a method for draining urine from a bladder using a urinary balloon catheter assembly, the method comprising:

a. installing a properly sized urinary balloon catheter comprising a port panel, wherein the port panel further comprises a magnetic click connector, in a suprapubic tract of a patient;
b. inflating the balloon to securely retain the balloon catheter in the suprapubic tract for an indefinite period of time until a sufficient amount of urine is collected in the bladder;
c. connecting a connecting line comprising a magnetic click connector to the port panel comprising the magnetic click connector by bringing the click connector of the connecting line in close proximity to the click connector of the port panel to allow the two magnetic click connectors to engage and securely connect together to form a water tight seal; and
d. draining the urine from the bladder through the balloon catheter and connecting line using an electrical-mechanical pump or a bulb suction device; or drainage by gravity.

As used herein, a properly sized balloon catheter is a low-profile catheter that is selected with an appropriate size to be securely retained upon installation in a suprapubic tract such that the balloon catheter remains secure during use over an extended period of time, such as for 1 week, 2 weeks, 3 weeks or about a month without requiring the replacement of the balloon catheter.

In one variation, the suprapubic tract is a previously established suprapubic tract. As employed herein, the balloon catheter may be properly sized to span from the skin to the bladder, where the retention balloon will hold the catheter in place. As used herein, a patient is a human patient or an animal, such as a dog, cat or other mammals.

In another aspect of the above method, the balloon catheter assembly comprises:

a) a balloon catheter comprising a proximal end, a distal end, wherein the balloon catheter further comprises:
a catheter wall defining a catheter lumen;
an inflatable balloon comprising a proximal end and a distal end, where the inflatable balloon is mounted at the distal end of the catheter and the catheter further comprises a lumen that extends from the balloon to the inflation valve and inflation port;
a base located at the proximal end of the catheter, wherein the base comprises a top end and a bottom end, wherein the base comprises an opening to the catheter lumen, and the base further comprising:
   i) a port panel comprising a top end and a bottom end, and a port extending therethrough, wherein the top end of the port panel comprises a magnetic click connector for connecting with a connecting line comprising a corresponding magnetic click connector, and wherein the bottom end of the port panel is attached to the top end of the base; and
   ii) an inflation port for inflating the balloon, where the inflation port is mounted on the base and the inflation port further comprises an inflation valve, where the inflation valve is in fluid communication with the balloon using an inflation lumen;

and

b) a connecting line comprising a proximal end and a distal end, wherein the proximal end comprises a first terminal magnetic connector for forming a water tight connection with the magnetic click connector on the port panel.

In another aspect of the method, the urine is drained into a drainage bag or drained directly into a toilet or disposal container. In another aspect, the method further comprises disconnecting the two magnetic click connectors, cleaning the surfaces of the magnetic connectors, and optionally, plugging the safety plug. As disclosed herein, the one-way valve may also prevent leakage.

As provided herein, the magnetic click connector is designed to allow a user with limited hand and/or finger coordination to readily connect and join the catheter to a connecting line and establish a secure and leak-free connection, by simply bringing the connecting line to close proximity to the base of the catheter. In addition, the click connector is also designed to allow quick and ease of disconnection of the catheter from the connecting line once the drainage procedure is completed. As disclosed herein, such balloon catheter as disclosed herein may be changed on a weekly or monthly basis as needed.

In one variation, the balloon catheter is an enteral feeding device. In another variation, the balloon catheter is a urinary catheter.

In one embodiment, this invention is a balloon catheter that is capable of being clamped to both retain the catheter against axial movement and to occlude its inflation lumen but not its principal lumen, and a fixator for use with it.

In one embodiment, this specification provides the catheter itself; and in its second aspect, this invention provides a fixator for retaining the catheter against axial movement and occluding the inflation lumen but not significantly occluding the principal lumen of the catheter. In a third aspect, this invention provides methods for stomal catheterization employing the catheter and fixator of the present invention. As disclosed herein, the catheter and fixator may be used to prevent urinary incontinence by capping and maintaining continent until the user is ready to drain the bladder.

In another embodiment, the present application discloses a balloon catheter having a balloon near and surrounding one end thereof, a tubular body comprising two tubular walls defining two lumens: (a) a first tubular wall having outer and inner surfaces, the inner surface defining a principal lumen through which fluids can flow between the distal end and the other end, and (b) a second tubular wall having outer and inner surfaces, the inner surface defining an inflation lumen through which the balloon can be inflated, the inflation lumen being in fluid connection with the balloon, where the second tubular wall lies outside but adjacent the outer surface of the first tubular wall over at least most of the length of the body. In one aspect of the above catheter, the first and second tubular walls are unitary. In another aspect, the outer surface of the wall defining the principal lumen is configured or formed such that the catheter may readily be externally clamped against axial movement by a clamping force that does not significantly occlude the principal lumen. In another aspect, the outer surface of the wall defining the principal lumen is corrugated.

In another embodiment, the application discloses a fixator for the balloon catheter as recited above, where the fixator has a lower surface adapted to lie against the skin of a patient in whom the catheter is to be used and a passage therethrough formed by a wall perpendicular to the lower surface that is shaped to engage the catheter. In one aspect of the fixator, that part of the wall of the passage that would engage the outer surface of the wall defining the principal lumen of the catheter is configured to engage that outer surface and clamp the catheter against axial movement. In another aspect, that part of the wall of the passage that would engage the outer surface of the wall defining the principal lumen of the catheter is configured to engage that outer surface and clamp the catheter against axial movement without significant occlusion of the principal lumen of the catheter. In another aspect, that part of the wall of the passage that would engage the outer surface of the wall defining the principal lumen of the catheter is provided with one or more ridges or corrugations. In another aspect, the fixator also comprises an occluder that can extend into that part of the passage that would contain the outer surface of the wall defining the inflation lumen of the catheter to compress the inflation lumen to occlude it when the occluder is engaged. In yet another aspect, the fixator has an upper surface that is shaped to engage a cap that is further configured to engage an external medical device, such as a drain tube or a connection to a source of liquid nutrients, to provide a fluid connection from the device through the passage to the principal lumen of the catheter.

In one aspect of the above fixator, the lower surface of the fixator further comprise a spacer mounted on the surface of the fixator. In another aspect, the fixator comprises a ferrule, a cap or an adaptor with one or more magnetic members at the terminal end of the ferrule, cap or adaptor that is configured to engage a cap that is connected to a drain tube, and where the cap comprises one or more corresponding magnetic members to securely join together to form a water tight seal joining the lumen of the drain tube and the principal lumen. In another aspect, the fixator comprises a ferrule, cap or adaptor with one or more magnetic members at the terminal end of the ferrule, cap or adaptor that is configured to engage a proximal end of a drain tube comprising one or more corresponding magnetic members to form a water tight or fluid tight seal with the ferrule, cap or adaptor.

In another aspect, the catheter comprises the recited fixator, wherein the catheter is a suprapubic urinary catheter, a gastrostomy tube (“G-tube”) or a jejunostomy tube (“J-tube”). In another aspect, the catheter is a suprapubic urinary catheter. In a particular aspect, the catheter is a gastrostomy tube (“G-tube”). In yet another aspect, the catheter is a jejunostomy tube (“J-tube”). In yet another aspect, the device may be an ACE-malone continence enema device that allows the use of the device in other location or portions of the GI system. In another variation, the catheter may be placed in other locations or portions of the GI system for removal or for installation of fluids, medication, nutritional fluids or other fluids.

In another embodiment, the application discloses a method for stomal catheterization of a patient comprising: (a) inserting a catheter as recited above, balloon end first, through a stoma into a body cavity or organ of a patient, (b) inflating the balloon of the catheter through the inflation lumen, (c) engaging a fixator as recited above around the catheter to secure the catheter axially, and (d) occluding the inflation lumen of the catheter by engaging an occluder within the fixator. In one aspect of the above method, the stomal catheterization is suprapubic urinary catheterization. In another aspect, the stomal catheterization is catheterization of the stomach or jejunum. In another aspect, the method further comprising cutting the catheter at the upper surface of the fixator and engaging a medical device thereto. In one aspect of the method, the liquid or urine from the bladder may be drained before performing the stomal catheterization.

In another aspect of the above disclosed magnetic click connectors, the medical device is a balloon catheter recited in each of the above embodiments, aspects and variations. In another aspect of the above, the catheter is an AMT Mini One® Balloon Button and the Kimberly-Clark Mic-Key Balloon Button that are further modified to include the elements and features as disclosed herein.

Certain embodiments of this invention are characterized by the specification and by the features of the claims of this application as filed.

### BRIEF DESCRIPTION OF THE FIGURES

FIGS. 1 through 15 illustrate certain embodiments of the invention.

FIG. 1 is a representative perspective view of the balloon end and a portion of the body of a balloon catheter according to this invention showing an uninflated balloon and an open and unclamped fixator body.

FIG. 2 is a perspective view of the balloon catheter of FIG. 1 showing an inflated balloon, and the fixator body positioned at a desired point along the length of the catheter.

FIG. 3A is a perspective view of the balloon catheter of FIG. 1 showing an inflated balloon with the fixator body engaged around the catheter at a desired point.

FIG. 3B is a top view of the balloon catheter of FIG. 3A showing the positioning of the catheter within the fixator body and the occluder.

FIG. 4A is a perspective view of the balloon catheter of FIG. 1 showing an inflated balloon with the catheter and fixator where the occluder of the fixator is engaged.

FIG. 4B is a top view of a balloon catheter of FIG. 4A showing the occluder in its engaged position.

FIG. 5A is a perspective view of a balloon catheter of FIG. 1 showing the occluder in its engaged position with a cylindrical fitting on the upper surface of the fixator body.

FIG. 5B is a top view of the balloon catheter of FIG. 5A showing the occluder in its engaged position with a cylindrical fitting on the upper surface of the fixator body.

FIG. 6 is a photograph showing the top view of a fixator with an alternative design of the occluder.

FIG. 7 is a photograph of a fixator clamped onto a short length of catheter tubing.

FIG. 8 is a photograph of a fixator where the catheter has been cut even with the upper surface of the fixator body and the cylindrical fitting placed on the upper surface of the fixator body.

FIG. 9 is a photographic representation showing the top view of an alternative design of a fixator in the unhinged and open configuration. The fixator incorporates a spacer on the lower surface of the fixator, where the spacer is shown as a concentric element, and in one variation, where the spacer is designed as a “4-bladed” radiation warning symbol.

FIG. 10 is a photograph of an alternative embodiment from a bottom view of a fixator clamped onto the balloon catheter, where the fixator incorporates a spacer on the lower surface of the fixator represented as a concentric element, and in one variation, where the spacer is designed as a “4-bladed” radiation warning symbol.

FIG. 11 is a photograph of an alternative embodiment of a fixator with a spacer having two spacer blades, showing the fixator clamped onto the balloon catheter, where a cylindrical fitting cap comprises 4 magnetic members, an opening and a one-way valve, where the cylindrical fitting cap is engaged onto the fixator, and where the cylindrical fitting cap further comprises 4 magnetic members that may be click connected to a drain tube comprising a corresponding connection with 4 corresponding magnetic members.

FIG. 12A is a drawing of an alternative embodiment showing a side view of a drainage line (or tubing) coupled to a drainage line cap and a fixator with a spacer.

FIG. 12B is a drawing of an alternative embodiment showing a side view of a drainage line (or tubing) engaged with or coupled to a drainage line cap that is capped onto (or screwed onto) a fixator with a spacer.

FIG. 13 is a photograph of one embodiment of the balloon catheter that has been clamped onto a fixator that comprises a spacer, where 1) the catheter has been cut; 2) a cylindrical fitting cap comprising magnetic members that has been click connected to a right angle elbow connector that is attached to a drain tube with a suction bulb and sump valve for withdrawing liquid or urine.

FIG. 14A is a drawing of a perspective view of one embodiment of the magnetic click connector and connecting line showing one embodiment of the magnetic click connector, where the magnetic members are shown as beads or pellets that are mounted on a base. The magnetic click connector may have a protrusion for engaging with a valve in the port to form a water tight connection. The base of the magnetic click connector is attached to a connecting line or tubing via a connector or adaptor.

FIG. 14B is a top view of one embodiment of the magnetic click connector showing the magnetic members that are mounted on a base. In one embodiment, the four magnetic members are shown as round beads.

FIG. 14C is a side view of a low profile balloon catheter and an inflated balloon mounted on the distal end of the balloon catheter, a base mounted at the proximal end of the catheter, the magnetic members mounted on the base and port panel comprising a port that is in fluid communication with the catheter lumen, an inflation port for inflating the balloon through the inflation lumen.

FIG. 15A is a cut out side view of a representative embodiment of a spring valve 290 showing an “I”-shape (or “T”-shaped) member 292 that may be displaced by the protrusion upon connection, such that fluids may flow through the hole or opening in the valve.

FIG. 15B is a cut out side view of a representative of an alternative embodiment of a spring valve 290 showing an “I”-shape (or “T”-shaped) member 292 that may be displaced by the protrusion upon connection, such that fluids may flow through the hole or opening in the valve.

FIG. 15C is a cut out side view of a representative embodiment of a flap valve 295 comprising a flap 297 that covers the hole or opening 299 in the valve.

FIG. 15D is a cut out side view of an alternative representative embodiment of a flap valve 300 comprising a flap 297 in the open configuration, which when closed, covers the hole or opening 299 in the valve.

### DETAILED DESCRIPTION OF THE INVENTION

### Definitions

“Balloon catheter” has its usual meaning, that is, a catheter having a balloon near and/or surrounding one end (e.g. the “distal end”) thereof, a tubular body comprising at least two lumens including a first lumen, referred to in this application as the “principal lumen”, through which fluids (e.g. urine if the catheter is used as a suprapubic or other urinary catheter, nutritional solution if the catheter is being used as a feeding tube) can flow between the distal end and the other end, the proximal end, of the catheter; and a second lumen, referred to in this application as the “inflation lumen”, through which the balloon can be inflated. In one aspect, the balloon may be a pre-formed, compliant balloon or deformable balloon, e.g., comprising a silicone balloon that may have a thickness of about 0.012 to 0.030 inches. The size of the balloon may be about 3 cc to about 75 cc.

“Comprising” or “containing” and their grammatical variants are words of inclusion and not of limitation and mean to specify the presence of stated components, groups, steps, and the like but not to exclude the presence or addition of other components, groups, steps, and the like. Thus “comprising” does not mean “consisting of”, “consisting substantially of”, or “consisting only of”.

A “magnetic member” means that a piece or a unit that produces a magnetic field and provides a force that pulls on other ferromagnetic materials, such as iron, which, for the purpose of this application, may also be considered a magnetic member. A magnetic member may also be a piece or a unit that is a magnet that attracts other magnets. As used herein, a “piece” or a “unit” may include one or more pieces of different sizes and configurations, including one or more pellets, one or more linear or curvilinear bars, discs or rods, and one or more circular or semi-circular units or annular units. Such magnets may include neodynium iron boron (NdFeB) magnets, samarium cobalt (SmCo) magnets, Alnico magnets, and ceramic or ferrite magnets. Alternatively, such magnets may include electromagnets.

The Catheter

In a first aspect, this specification discloses a balloon catheter that is capable of being clamped to both retain the catheter against axial movement and to occlude the inflation lumen but not the principal lumen. To achieve this, the catheter has the wall defining the inflation lumen completely outside the wall defining the principal lumen over at least most of the length of the body. Typically, the wall defining the inflation lumen is thinner than the wall defining the principal lumen so that it is easier to deform the wall defining the inflation lumen to occlude the inflation lumen than to deform the wall defining the principal lumen in a way that significantly occludes the principal lumen. The walls defining each of the principal and inflation lumens may be made from any suitable flexible polymer, including biocompatible polymers, such as silicone, latex, polypropylene, polyethylene, polyurethane or reinforced silicone; and suitable materials for the construction of urinary catheters and feeding tubes are well known to those of ordinary skill in the art. Optionally, the catheter may be coated by antibiotics, silver or any other suitable antimicrobial material to minimize infection and encrustation. The reinforced silicone tubing that may form the catheter wall, may have substantially the same thickness as a non-reinforced silicone tubing, and may have the same modulus as a non-reinforced silicone tubing, but the walls of the reinforced tubing will not kink or collapse during placement, insertion, use and removal. Typically, further, the outer surface of the wall defining the principal lumen will be formed such that the catheter may readily be externally clamped against axial movement by a clamping force that does not significantly occlude the principal lumen: a convenient outer surface of the wall defining the principal lumen for that purpose will be one that has corrugations transverse to the catheter axis; the corrugations can engage with corresponding corrugations, notch or indentation on a fixator to limit axial movement of the catheter with respect to that fixator without requiring the fixator to exert such force as to significantly occlude the principal lumen. The corrugations may be of any desired shape that may cooperate with a fixator to prevent axial movement of the catheter, such as sinusoidal or sawtooth; or the outer surface of the wall defining the principal lumen may be generally smooth but be transversely grooved, for example with a groove cross section that is generally rectangular or that is somewhat wedge-shaped, with one wall of the groove perpendicular to the outer surface of the wall defining the principal lumen and the other at an obtuse angle to the outer surface: the perpendicular surface of the grooves facing the balloon end of the catheter to provide maximum resistance to movement of the catheter axially into the body when one of more of these corrugations or grooves is engaged by an external fixator, as discussed further.

The size of the catheter may be from about 6 to about 86 Fr, 8 to 36 Fr, or may be about 12 to 26 Fr (French scale or French gauge—the French size is three times the diameter in millimeters), such as from 12 to 14 Fr, or 16 to 20 Fr, and for example, may be about 15 Fr, 16 Fr, 17 Fr, 18 Fr or about 19 Fr.

Typically, for adolescent patients, a catheter size of 14 FR may be used, and for younger children, pediatric catheter sizes of 6 to 12 Fr may be used. The length of the catheter may range from about 2 to 55 cm, about 4 to 22 cm, or about 15 to 55 cm; or may be about 14 to 15 inches, or about 35.6 to 38.1 cm. In one variation, the length of the catheter, including the terminal end, may be about 40 to 45 cm, or about 43 cm.

The Fixator

In its second aspect, this invention relates to a fixator or retention device for the catheter of the first aspect of this invention. In use, the lower surface of the fixator will lie against the skin of the patient into whom the catheter has been inserted, and the fixator will engage the catheter to clamp the catheter against axial movement, particularly against axial movement of the catheter into the patient's body. The fixator will also be capable of occluding the inflation lumen of the catheter without significantly occluding the principal lumen. The fixator may be made out of any suitable relatively rigid polymer or plastic, including biocompatible polymers, such as silicone, polyolefin (polyethylene, polypropylene, or the like), polyurethane, polyether block amide, nylon, polycarbonate or the like; and suitable materials for the construction of the fixator will be well known to those of ordinary skill in the art. The fixator is generally disc-shaped, typically circular but may be oval or oblong shaped, and having a diameter of between 1 and 20-cm, or about 3 to 6 cm, such as about 3 cm. An exemplary design is one in which the axial center of the fixator coincides with the axial center of the principal lumen of the catheter with which it is used. Since the fixator is required to be capable of clamping around the catheter, it will be split along a diameter also containing the axial center of the inflation lumen into two halves when open, the halves being hinged together, such by a conventional hinge or a living hinge, with the barrel portions formed in each half and an external pin passing through the barrel (as is shown in the figures, as an example) or by the two halves being integrally linked by a flexible connection. The two halves will desirably have alignment structures formed in the straight surface (the diametral surface) of each half to engage when the fixator is clamped around the catheter and align the halves with respect to each other. In the figures showing a representative embodiment, this is shown as a “mortise and tenon” arrangement, with one half of the fixator having two projections, one on each side of the axial center, and the other half having corresponding recesses to accommodate those projections. The projections and recesses may, if desired, be designed to “snap” together, such as by the projections having a groove parallel to the axial surface and the recesses having a ridge that engages the groove when the fixator is clamped around the catheter; or another closure method or mechanism may be used to hold the fixator closed when in use. In the figures, the fixator is held closed by a fitting that engages the body of the fixator around the axis of the fixator body, as will be discussed in more detail with respect to the figures. Other closure mechanisms that may be employed include a latch, an adjustable or non-adjustable slide bolt lock, a flip latch, cane bolt latch, a hasp latch or a hook latch, for example. Thus the fixator has a passage therethrough formed by a wall perpendicular to the lower surface that is shaped to engage a catheter of the first aspect of the invention, and that part of the wall of that passage that would contain the outer surface of the wall of the principal lumen of the catheter is configured to engage that outer surface and clamp the catheter against axial movement but without significantly occluding that principal lumen. So, for example, if the outer surface of the wall of the principal lumen has corrugations or grooves transverse to the catheter axis, the wall of the passage will have a transverse ridge or ridges or corrugations extending into the passage to engage the grooves or corrugations on the outer surface of the wall of the principal lumen. The fixator also has an occluder that can extend into that part of the passage that would contain the outer surface of the wall of the inflation lumen of the catheter, so that when the occluder is engaged, it compresses the inflation lumen to occlude it and prevent fluid flow to and from the balloon, thereby, for example, preventing the balloon of the catheter from deflating.

Desirably, the fixator is shaped, e.g. at its upper surface (i.e. the surface that does not lie against the skin when in use), to engage a cap, such as a continence cap, or an adaptor that may engage a suitable external medical device, such as a drain tube 257 when the catheter is being used as a suprapubic urinary catheter, or a connection to a source of liquid nutrients when the catheter is being used as a feeding tube, to provide a fluid connection from the device through the passage to the principal lumen of the catheter. The cap may be configured with an internal compression fitting, or configured with threads 262, such that when the cap is engaged onto the fixator, the ferrule, which may be built into the cap, radially compresses and firmly holds the catheter onto the fixator and prevents the catheter from being moved or dislodged. In one variation, the cap may be engaged onto or connected to the fixator by one or more corresponding magnetic members as described below. In one aspect, the fixator may comprise a one-way valve, such as a dome valve, such that when a drain tube that comprises a male member or protrusion 260, is connected to the fixator, urine or fluid may properly drain out of the catheter. In one variation, a suction bulb 265 may be used to suction or draw out the urine or fluid.

In one variation, a spacer may be mounted on the lower surface of the fixator such that the spacer provides a soft, non-irritating surface when it is in contact with the skin. The spacer may be of substantially the same, smaller, larger or similar shape as the fixator, and it may be a ring, a broken ring, a disc or a set of discs having, for example, concentric elements that is similar in design to a “4-bladed” radiation warning symbol. In one variation, the spacer may be configured as a 2-, 3-, 4- or 5-blades or up to 20 blades, with regular or irregular size of spacing between the blades. In one variation, the blades may be dental shaped, oval shaped, blade shaped or pie shaped; and may project or be raised above the surface of the fixator, such as by about 2 mm, 3 mm, 4 mm or more to provide sufficient ventilation for the skin. In one aspect, the spacer may be mounted on the fixator and raised up from the fixator to provide some air ventilation between the gaps of the blades and also ventilation between the spacer element and the skin. The spacer may be made of a soft material, such as a soft plastic, including for example, methyl methacrylate, ethyl acrylate, ethyl vinyl acetate, silicone, acetal, acrylic, polyimide, ECTFE, nylon, EPT, polycarbonate film, polyester film, polypropylene and PVDF. In one variation, there is provided a seal, such as a protective shield that may be placed between the lower surface of the fixator and the skin for protecting the skin and eliminate or minimize any potential leakages. The shield may be of the same or different footprint or shape than that of the fixator and may be reversibly fixed and removed, via an adhesive.

In a third aspect, the upper surface of the fixator comprises an adaptor comprising one or more magnetic members for engaging an adaptor connected to the proximal end of a drain tube, wherein the adaptor on the proximal end of the drain tube comprises one or more corresponding magnetic members to engage or connect with the one or more magnetic members on the adaptor on the upper surface of the fixator to form a water tight or fluid tight seal. In one variation, the adaptor on the proximal end of the drain tube is fitted with an elbow connection such that the drain tube is connected at substantially 90 degrees, 75 degrees, 50 degrees or about 45 degrees, to the adaptor on the upper surface of the fixator. The elbow connection may be rotatably mounted such that the drain tube may be rotated into a convenient location or direction when in use. In one variation, the elbow connection may be connected with a swivel to allow rotation of the elbow and tubing. In another variation, the adaptor on the proximal end of the drain tube is fitted with straight or linear connection such that the drain tube is connected straight out or linear (i.e., at about 180 degrees) with the adaptor on the upper surface of the fixator. Accordingly, when the one or more magnetic members mounted on the adaptor are placed in close proximity to the one or more magnetic members on the proximal end of the drain tube, the attracting magnetic force engage and connect the adaptor and the drain tube to engage or connect together to provide a fluid tight seal. In one variation, the connecting surfaces are provided by an elastomer seal, an elastomer coating, or one or two sealing O-rings to further provide an air-tight and/or water tight connection. The O-ring may be mounted inside, i.e., toward the inside lumen, of the magnetic members or outside of the magnetic members toward the outer wall; or both inside and outside for a tighter seal. Once the fluids are drained, the removal of the drain tube from the adaptor and fixator may be readily performed by a breakaway function to break the connection when desired. The strength or tightness of the water tight connection for ease of connection and ease of disconnection may be controlled by the number, shape and configuration of the magnetic members mounted on the adaptor on the fixator and on the adaptor mounted on the drain tube. In one aspect, each of the corresponding magnetic members 256/258 may be configured as one or more pellets, such as 2, 3, 4, 5 or more pellets that may be rectangular or round in shape; or each of the corresponding the magnetic members 256/258 may comprise of one or more rods, such as 2, 3, 4, 5 or more rods, one or more semi circles, such as 2, 3 or 4 semi circles or a single circular ring; or a combination thereof. In one variation, the pellets, rods, semi-circle or circular rings, or combinations thereof, maybe configured as two or more concentric ring configuration to provide a stronger seal when connected together. As disclosed herein, one the magnetic members on the fixator and on the adaptor may be of similar or different structure or configuration. For example, the magnetic member on the fixator may be a circular disk, while the magnetic members on the adaptor may be a series of rods or pellets.

In one variation, the magnetic member(s) may be mounted on the surface of the adaptors, or may be incorporated into or under the (plastic) surface of the adaptors such that the magnetic member(s) are not exposed to air, or may be subject to oxidation or corrosion. Such simplicity of the self-locating, self-mating connectors and connecting mechanism, i.e., a click connect or a quick connect, provides a tremendous advantage to patients with limited manual dexterity and mobility, where the patient would not have to manually manipulate and connect and/or tighten any mechanism such as dials, clasps, rings, etc. . . . to engage and ensure a water tight seal or connection.

In one variation, once the click connection is made, one of the 2 sides of the connecting tubes or lines may be equipped or mounted with a reversible locking mechanism, such as a looping fastener, such as a ring or latch. To employ the fastener, the patient may lift the fastener from one side of the connecting tubes over the connection and mount the fastener onto the opposite side of the connection to engage with a collar or edge of the connecting line to further secure the connection. Such a fastener may be employed to further secure the magnetic click connector, for example, when a patient goes to sleep to minimize the risk of unintentionally dislodging the connection during sleep.

In one variation, a disconnecting frame comprising a lever, where the disconnecting frame is coupled to or connected to at least one fork, or two forks, and where the disconnecting frame may be mounted or clamped onto one of the terminal end of the adaptor on the fixator or on the adaptor that is mounted on the drain tube. When desired, the water tight connection may be readily disconnected or detached by simply pressing on the lever on the disconnecting frame that connected to the fork, which readily pries open or separate the water tight connection. In one variation, the fork(s) may be configured to latch between the connecting surfaces of the two ends of the magnetic members or the fork(s) may engage or latch on a ridge, collar or ear(s) of the one of the two terminal ends, for prying and disconnecting the connection. In one aspect, the adaptor comprises at least one or two vents 264 that is in fluid communication with the inflation lumen such that when the balloon is deflated, the liquid in the balloon may flow freely out of the vent or opening 264. In one variation, the vent(s) is placed on the side of the adaptor.

In use, the catheter is inserted, balloon end first, through a stoma into a body cavity or organ of a patient, such as into the bladder (suprapubic urinary catheter) or the stomach or jejunum (“feeding tube”), the balloon inflated to hold the balloon end firmly within the cavity or organ, and the catheter is withdrawn until the balloon is firmly against the inner surface of the cavity or organ. The fixator is then closed around the catheter to secure the catheter axially, and the inflation lumen of the catheter is occluded by external compression by an occluder within the fixator. In one variation, the inflation lumen may be occluded or blocked by using a plug or an adhesive; or a radial or circumferential compression may be applied. Alternatively, an axial occlusion of the lumen may be performed by inserting an object, such as a plug into the lumen. At that point, the catheter is secured between the fixator at the skin surface and the inflated balloon within the body cavity or organ, and the balloon is secured against deflation by the occlusion of the inflation lumen.

The catheter may then be cut at the fixator without the balloon becoming deflated or the catheter moving into the body through the stoma, so that the catheter provides a path from the body cavity or organ effectively to the skin surface; and the appropriate medical device (drain tube, source of liquid nutrients) is then attached to the fixator. When the catheter is no longer needed, after any medical device attached to the fixator is removed, the occluder is disconnected to allow the balloon to deflate, and the catheter and fixator can be removed, withdrawing the catheter from the stoma. Because the catheter may be deployed into a patient and then custom cut to the right size to accommodate patients of different waist sizes or abdominal girths, the catheter of the present invention may be considered a one-size fits all catheter. In another embodiment, the catheter may be constructed from two or three different intermediate lengths and then further custom cut to accommodate the significant differences in the waist size of the patient population. Alternatively, the catheter may be constructed of several different sizes or lengths, such as 10 or 20 different sizes or lengths to individually accommodate different patients without cutting the catheter.

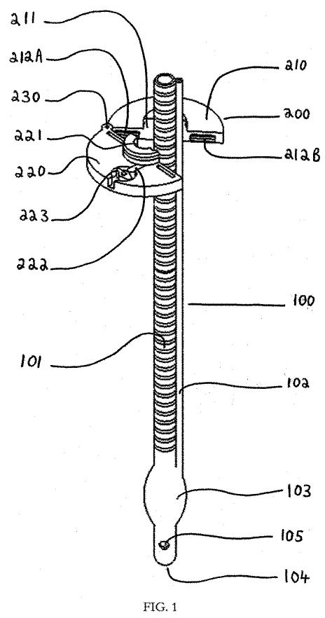

FIG. 1 is a perspective view of a balloon catheter according to the first aspect of this invention together with a fixator body according to the second aspect of this invention, where the balloon is uninflated (or deflated) and the fixator body is not clamped to the catheter. This represents the first stage of stomal catheterization using the balloon catheter of this invention, where the catheter has been inserted into a stoma in the body of a patient so that the balloon end (the distal end) of the catheter is received within a body cavity or organ, except that the body of the patient is not shown in the figures. Here the catheter shown generally at 100 comprises a wall 101 defining a principal lumen and a wall 102 defining an inflation lumen, wall 102 lying outside but adjacent to wall 101. The distal end of the catheter is shown having a balloon 103 around the wall 101 of the principal lumen, the balloon 103 being connected to the inflation lumen so that it may be inflated by the introduction of fluid into the inflation lumen from the proximal end (not shown). The wall 101 defining the principal lumen terminates in a tip 104 which is furnished with one or more apertures 105 to allow fluid to flow between the principal lumen and the outside of the catheter, such as between the principal lumen and the interior of a body cavity or organ, when the catheter is in use. Here the wall 101 is shown as provided with transverse corrugations on its outer surface, configured to engage a corresponding ridge on the fixator when the fixator is clamped onto the catheter and prevent axial movement of the catheter. While the proximal end of the catheter is not shown, it will be understood that the catheter may have any appropriate fittings at the proximal end to enable inflation of the balloon (an inflation port) and, for example, to introduce or remove fluid via a syringe, or to admit a guidewire down the principal lumen to the tip to guide the catheter when it is being introduced into the body of a patient through a stoma. The fixator body shown generally at 200 here comprises a split disc structure, having two halves 210 and 220 that are hinged together at 230, shown here as a barrel hinge. The disc is split down a diameter that includes, when the catheter is clamped in the fixator, the axes of the principal and inflation lumens of the catheter. Each half has a wall, 211 and 221 respectively, projecting axially outward from the body to, when closed together, clamp the catheter against axial movement. The fixator is also provided with an occluder to occlude the inflation lumen of the catheter when the occluder is engaged. Here this is shown as an occluder rod 222 sliding through a passageway in the fixator body half 220 to be able to apply pressure to the wall 102 of the inflation lumen when the fixator is clamped around the catheter and an occluder cam 223, rotatably mounted to that half 220, that in its non-engaged position (transverse to the occluder rod) does not urge the occluder rod against the wall of the inflation lumen. In this embodiment, the alignment structures formed in the straight surface (the diametral surface) of each half form a “mortise and tenon” arrangement, with the two projections 212A and 212B being visible in one half, though the corresponding recesses of the other half are not visible.

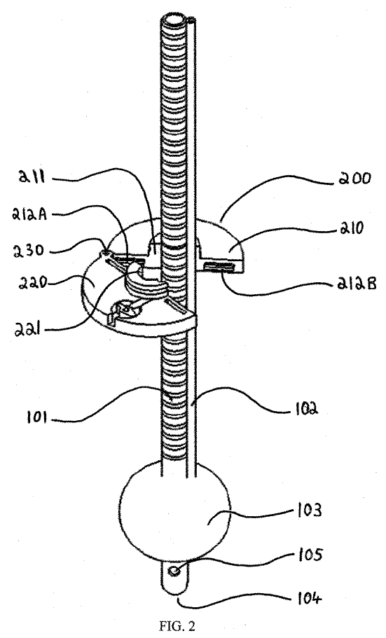

FIG. 2 shows the same catheter and fixator as in FIG. 1 from the same perspective except that the balloon 103 has now been inflated, and the fixator body positioned at the desired point along the length of the catheter. The balloon is inflated by fluid (e.g., sterile water) supplied through the inflation lumen through an inflation port at the proximal end of that lumen (not shown, and not claimed), and remains inflated because of pressure within the inflation lumen applied and maintained at the inflation port. This represents the second stage of stomal catheterization, where the catheter balloon is now positioned against the inner surface of the body cavity or organ and the lower surface of the fixator body is positioned against the skin above the stoma.

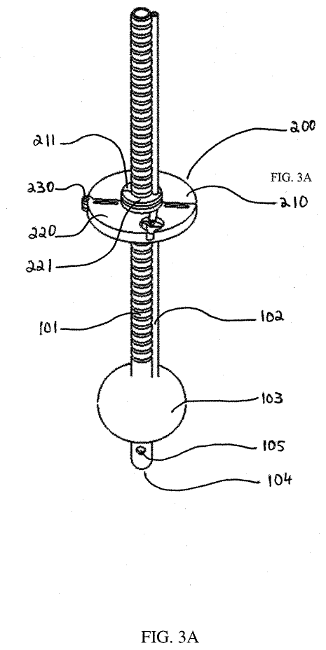

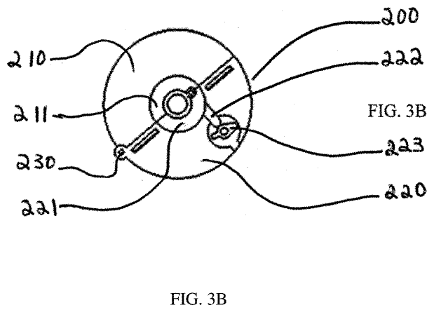

FIG. 3A shows the same catheter and fixator as in FIG. 1 from the same perspective except that the fixator body has now been engaged around the catheter at the desired point, thereby clamping the catheter against axial movement with respect to the fixator. This represents the third stage of stomal catheterization, where the catheter is now fully in place, but the occluder of the fixator is not yet engaged. FIG. 3B is a view from above, in which the positioning of the catheter within the fixator body and the occluder each may be seen more clearly.

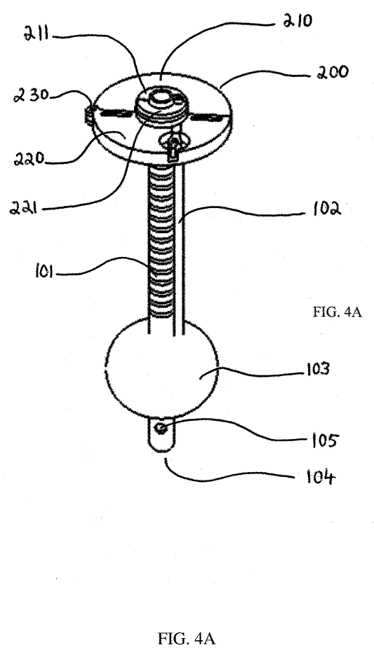

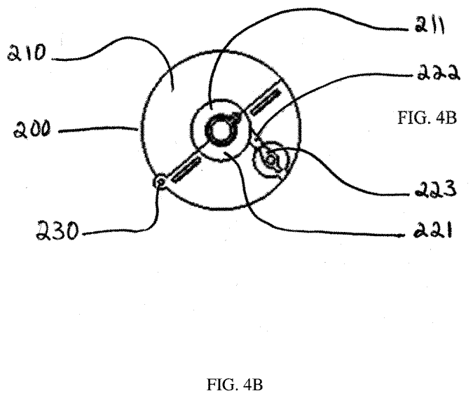

FIG. 4A shows the same catheter and fixator as in FIG. 1 from the same perspective except that the occluder of the fixator has now been engaged, occluding the inflation lumen so that the balloon of the catheter now remains inflated because of the occlusion of the inflation lumen, permitting removal of that part of the catheter extending beyond the fixator without deflation of the balloon. The part of the catheter extending beyond the fixator has now been cut off at the upper surface of the fixator. This represents the third stage of stomal catheterization, where the catheter is now fully in place and cut to length. FIG. 4B is a view from above, in which the occluder may be seen more clearly in its engaged position. Here the occluder cam 223 has been rotated in line with the occluder rod 222, urging the occluder rod 222 against the wall 102 of the inflation lumen. As disclosed in one embodiment, in the engaged position, the occluder rod 222 and occluder cam 223 lay in a ridge, slot or channel and are flushed with the upper surface of the fixator such that the occluder rod 222 and occluder cam 223 are not accidently touched or moved, resulting in a premature disengagement from its engaged position.

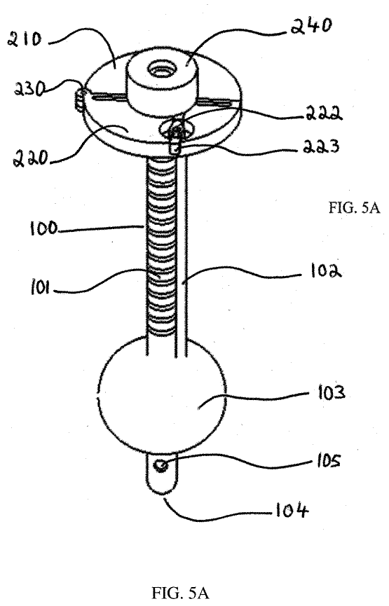

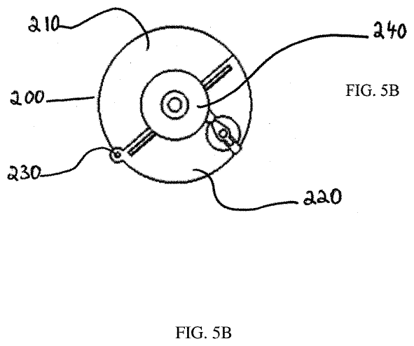

FIG. 5A shows the same catheter and fixator as in FIG. 1 from the same perspective except that a cylindrical fitting 240 has now been placed on upper surface of the fixator body. The fitting 240, which is a part of the fixator, serves two purposes: first, by engaging both halves 210 and 220 of the fixator body, it helps clamp the fixator tightly against the catheter, preventing axial movement of the catheter with respect to the fixator; second, the fitting may be shaped internally and/or externally to engage a medical device (e.g. a drainage line and bag for a urinary catheter, a source of liquid nutrients for a feeding tube) to the upper surface of the fixator.

In one embodiment when used as a urinary catheter for example, the drainage line 257 include a valve to allow urine drainage. The valve may include a drainage port that may hold a duckbill valve or any valve that stops the flow of urine, such as a ball valve, a diaphragm valve, a butterfly valve, a dome valve, an umbrella valve, a flap valve or a gate valve. The urinary bladder drainage system may include a receptacle that transfers urine into a collection device. In one embodiment of the bladder drainage system, there is included a disposable receptacle that may include a connector, a tubing with a cap and a reservoir. The receptacle may be configured to couple to a valve. The connector may couple to a drainage port to allow bladder drainage. The union may be achieved by mechanical or magnetic mechanisms. In one embodiment of the bladder drainage system, the tubing with a cap may be used to transfer urine from the valve to the reservoir 265. Urine may be aspirated or sucked out by squeezing the reservoir or drained by gravity. The reservoir 265 may be preferably configured to stop the suction after all the urine is drained. The receptacle may be made out of plastic, silicon, or latex. Alternatively, disposable receptacle may be made out of any suitable material to transfer urine into a collector.

The fitting 240, and those parts 211 and 221 of the fixator body with which it engages, are preferably designed to “snap” together to prevent accidental disengagement, such as by having one of their cooperating surfaces having a transverse groove and the other having a transverse ridge. This represents the fourth stage of stomal catheterization, where the catheter is now fully in place and ready for connection to the appropriate medical device. FIG. 5B shows the same view as FIG. 4B except that the fitting now covers the cut end of the catheter.

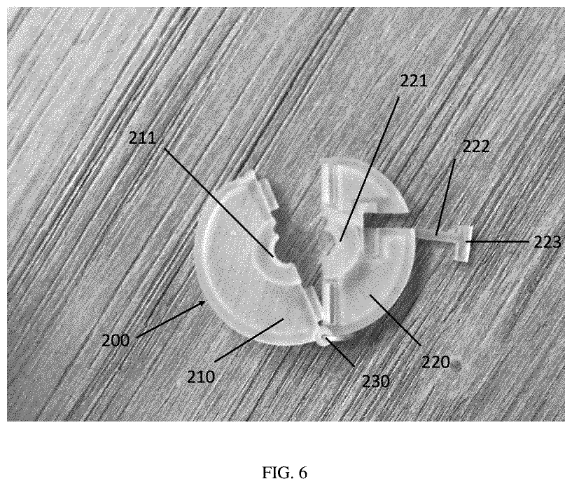

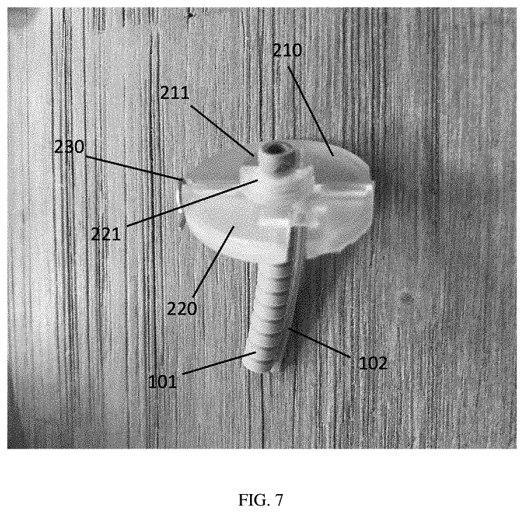

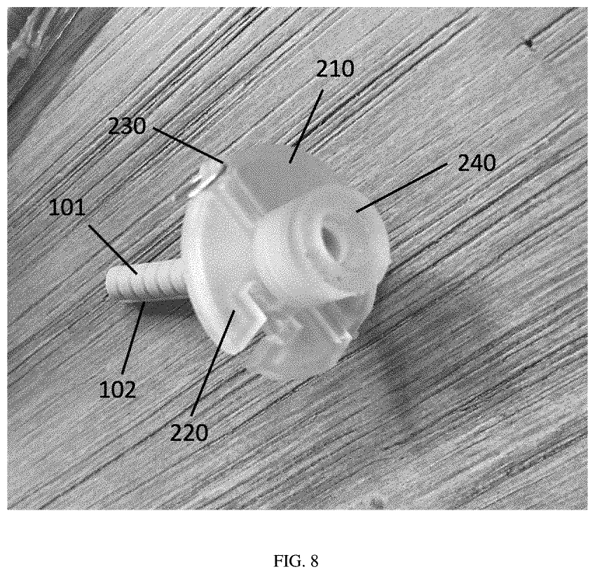

FIG. 6 is photograph showing an alternative fixator according to the second aspect of this invention. The fixator is similar to that shown and described in FIGS. 1 through 5B except for the design of the occluder. In this view from above, the halves of the two passageways through the body of the fixator for the two walls of the catheter can readily be seen, as can the alignment projections on one of the halves. In this and in FIGS. 7 and 8, the occluder is a cylindrical rod with a crossbar at one end, visible in the picture to the side of the occluder body, and the half of the fixator body with which it cooperates has a passageway therethrough intersecting with the diametral surface of that half at the axis of the inflation lumen, and a keyway extending from that passageway to the circumferential surface of that half. The keyway also includes, at its inner end, at least one recess in the fixator body half perpendicular to the keyway that is capable of receiving the crossbar of the occluder. Once the fixator has been clamped around the catheter, the occluder is pushed down the keyway so that the rod enters the passageway and its end presses against the wall of the inflation lumen to occlude the inflation lumen. The occluder can then be rotated along the axis of the rod so that the crossbar is received in the recess(es) in the fixator body half and the occluder is retained in its engaged position. When it is desired to open the inflation lumen to allow the balloon to deflate, the occluder is rotated to release the crossbar from the recess(es) and the occluder is withdrawn down the keyway.

FIG. 7 is a photograph showing the fixator of FIG. 6 clamped onto a short length of catheter tubing. The occluder of the fixator is engaged to occlude the inflation lumen of the catheter tubing, and the keyway in the fixator body half along which the occluder rod is inserted may be seen. This view is analogous to FIG. 4A.

FIG. 8 is a photograph showing the fixator of FIG. 7 where the catheter has been cut even with the upper surface of the fixator body and the cylindrical fitting placed on the upper surface of the fixator body. This view is analogous to FIG. 5A.

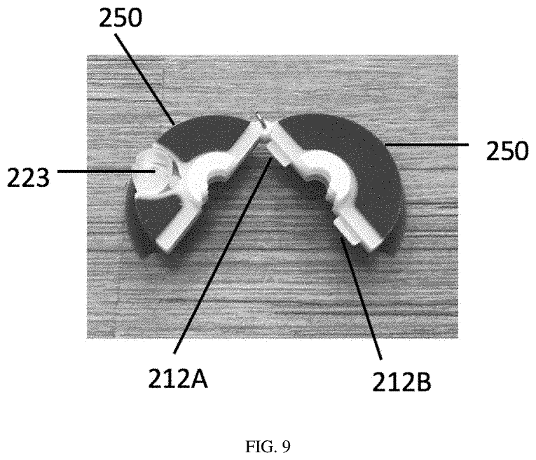

FIG. 9 is a photograph representation showing the top view of an alternative design of a fixator in the unhinged and open configuration, that incorporates a spacer 250 on the lower surface of the fixator represented as a concentric element, where two blades 252 of the spacer 250 are visible and designed as a “4-bladed” radiation warning symbol. In one variation, the spacer is a disc that has no blades, and accordingly, no gaps.

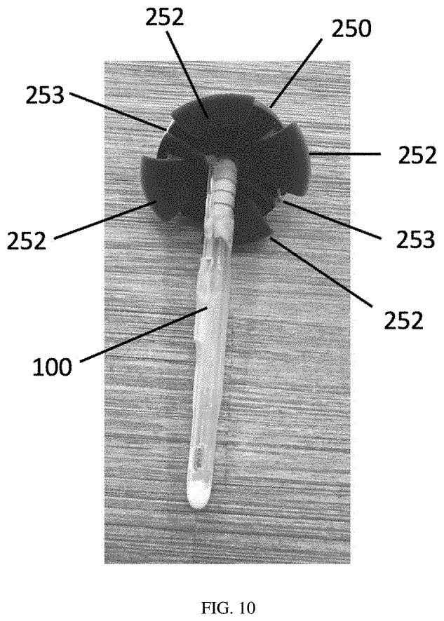

FIG. 10 is a photograph of an alternative embodiment from a bottom view of a fixator 200 clamped onto the balloon catheter 100, where the fixator that incorporates a spacer 250 on the lower surface of the fixator represented as a concentric element, where the 3 (of 4) spacer blades 252 are visible and are designed, for example, as a “4-bladed” radiation warning symbol. The gaps or openings 253 between the spacer blades 252, may include for example, 4 gaps between 4 blades, or 2 gaps, 3 gaps, 4 gaps, or more, and may allow air flow or ventilation between the skin and the fixator, and provides a comfortable and non-irritating contact surface with the skin. The spacer 250 may be built in or be a part of the fixator, or the spacer may be an independent element (i.e., un-connected to the fixator) that may be placed under the fixator, similar to a washer. In one variation, the spacer 250 may be made of a soft, non-irritating plastic, and may have a flexible concave undersurface to comfortably adapt to the skin. A protective shield (not shown) may be placed between the skin and the spacer 250.

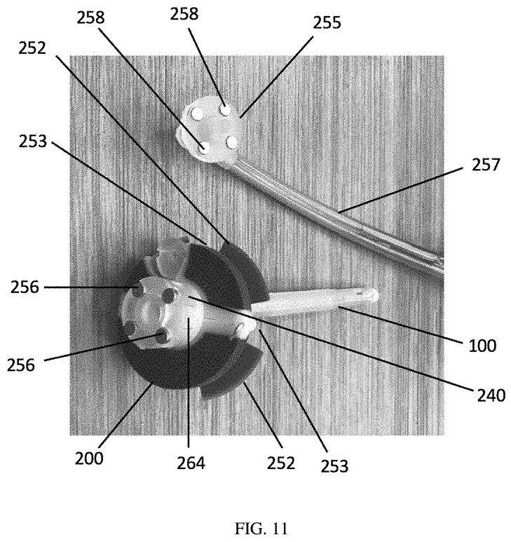

FIG. 11 is a photograph of an alternative embodiment of a fixator 200 with a spacer 250 with two spacer blades 252, showing the fixator 200 clamped onto the balloon catheter 100, where a cylindrical fitting cap comprising 4 magnetic members 256, an opening and a one-way valve, and where the cylindrical fitting cap 240 is engaged onto the top of the fixator 250, and where the cylindrical fitting cap 240 further comprises 4 magnetic members 256, that may be click connected to the corresponding fitting cap 255 connected to a drain tube 257 and comprising 4 corresponding magnetic members 258.

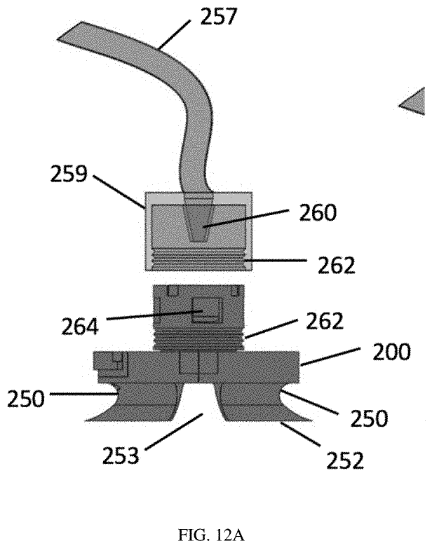

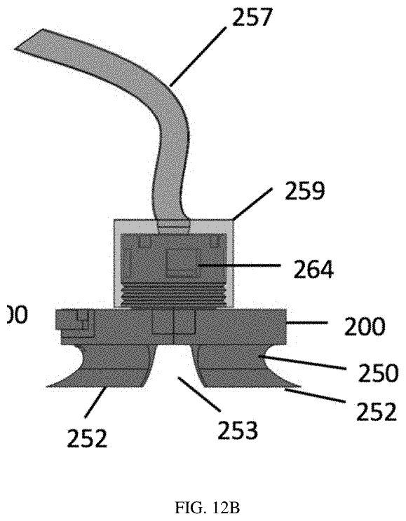

FIG. 12A is a drawing of an alternative embodiment showing a side view of a drainage line (or tubing) 257 coupled to a drainage line cap 259 and a fixator 200 with a spacer 250. The spacer 250, shown with 2 exposed spacer blades 252 (with other 2 blades not visible) may be engaged onto or attached to the bottom surface of the fixator body 200.

FIG. 12B is a drawing of an alternative embodiment showing a side view of a drainage line (or tubing) 257 engaged with or coupled to a drainage line cap 259 that is capped onto (or screwed onto) a fixator 200 with a spacer 250. The drainage line cap 259 is shown with a vent opening 264 for draining the liquid from the balloon of the balloon catheter.

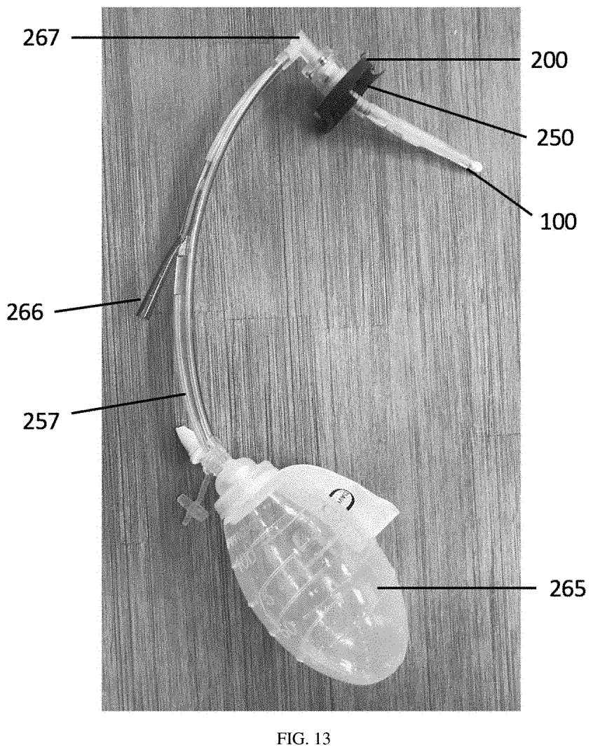

FIG. 13 is a photograph of one embodiment of the balloon catheter 100 that has been clamped onto a fixator 200 comprising a spacer 250, where 1) the catheter has been cut; and 2) a cylindrical fitting cap comprising magnetic members that has been click connected to a right angle elbow connector 267 that is attached to a drain tube 257 and a suction bulb 265 and sump valve 266 for withdrawing liquid or urine. Depending on the suction or vacuum that may be generated by the suction bulb 265 for drawing out liquid, optionally, a sump valve 266 may be incorporated with the drain tube. As employed herein, a sump valve may be a pressure sensitive check valve, a one-way valve or flapper style valve, such that the valve may close off when the pump pulls a particular pressure to avoid any tissue damage from high pressure or vacuum.

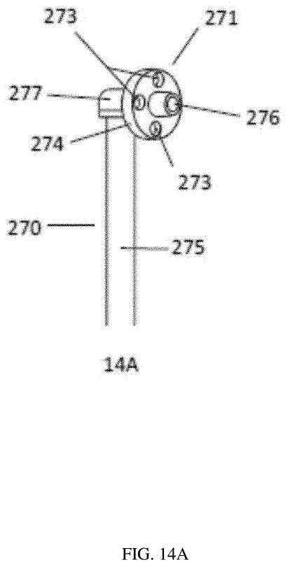

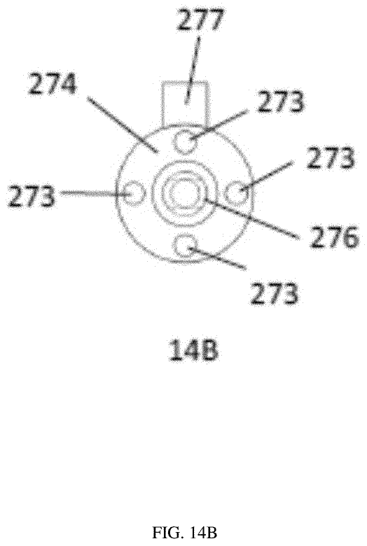

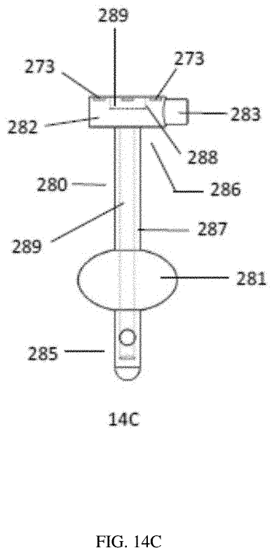

FIG. 14A is a drawing of a perspective view of one embodiment of the magnetic click connector and connecting line 270 showing one embodiment of the magnetic click connector 271, where the magnetic members 273 are shown as beads or pellets that are mounted on a base 274. The magnetic click connector may have a protrusion 276 for engaging with a valve in the port to form a water tight connection. The base of the magnetic click connector is attached to a connecting line or tubing 275 via a connector or adaptor 277, here, a right angle elbow adaptor or connector.

FIG. 14B is a top view of one embodiment of the magnetic click connector 271 showing the magnetic members 273 that are mounted on a base 274. In one embodiment, the four magnetic members 273 are shown as round beads.

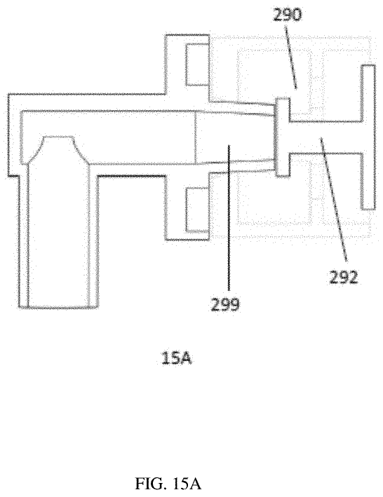

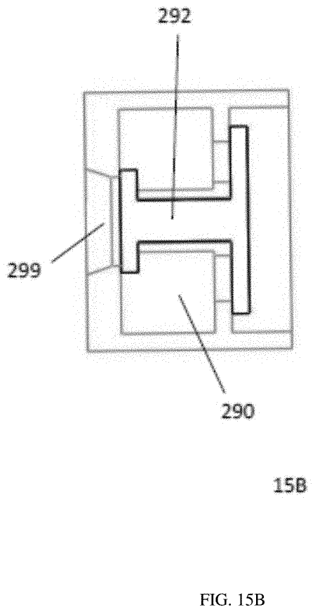

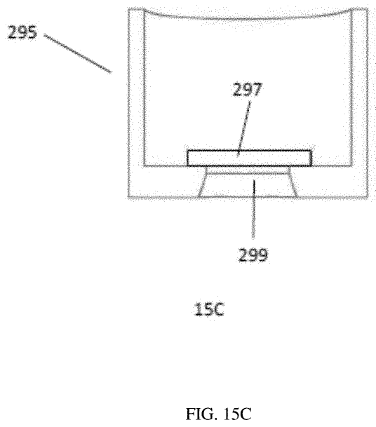

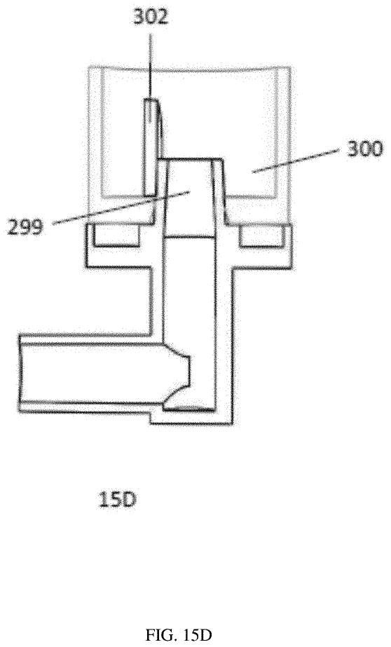

FIG. 14C is a side view of a low profile balloon catheter 280 and an inflated balloon 281 that is mounted on the distal end 285 of the balloon catheter 280, a base 282 that is mounted at the proximal end of the catheter 286, the magnetic members 273 mounted on the base 282 and port panel 288 comprising a port 288 that is in fluid communication with the catheter lumen 289, an inflation port 283 for inflating the balloon through the inflation lumen 287. In one variation, the port panel may be rotatably mounted on the base. The base 282 may contain a valve (not shown in FIG. 14C, but shown in FIG. 15A-D) suitable for forming a water tight seal to prevent fluid from passing through the catheter lumen 289 and/or leaking out of the port 288. The valve may be adapted to seal at low pressures and/or when nothing is crossing the valve. For example, the valve may comprise a resiliently self-closing means. The valve may be further adapted to seal at pressures between 0 and 250 cm of water. Ideally the valve has a relatively small height dimension, e.g., to enable the base 282 to be relatively thin, e.g., between about 0.25 and 2 cm or less than 1 cm. The valve may be opened by engagement of a protrusion 276 present on a mating base such as the one shown in FIG. 14A. The protrusion 276 may engage with the base 282 such that the protrusion enters the port 288 and further engages the valve (not shown in FIG. 14C, but shown in FIG. 15A-D). The protrusion 276 may at least partially open the valve such that fluid may flow through the port and into the tubing 275. The protrusion 276 may itself form a seal with the valve (not shown in FIG. 14C, but shown in FIG. 15A-D). Alternatively or in addition, the base 282 and/or the port 288 may engage with a mating surface or feature on the base of the magnetic click connector 271. In one aspect, the force of attraction between the base 282 and/or port 288 and the magnetic click connector 271, each of which may comprise one or more magnetic member may be such that the elements are attracted into a mating configuration from a distance of between 0-5 cm and from an angle of approach of between 0-45 degrees. In one embodiment, the protrusion 276 have a length between 1 to 15 mm, i.e., so as to easily engage and come into alignment with the port 288 and/or valve (not shown in FIG. 14C, but shown in FIG. 15A-D) from an angle of approach. In another aspect, the peak force of attraction may be between 100 grams to 3,000 grams force. Further, the force of engagement of the protrusion 276 with the valve (not shown in FIG. 14C, but shown in FIG. 15A-D) may be less than the peak force of attraction, e.g., such that if the base 282 is positioned in relative proximity to the magnetic click connector 271, the elements will be attracted to one another such that the valve is opened to fluid flow and a water tight seal is enabled.

Further with regard to the protrusion 276 may be annular or hollow, such that fluid flows through the protrusion. Alternatively, it may be solid, serving primarily to splint open the valve while permitting fluid to flow around it. In this case a water tight seal may be created between elements of the valve (not shown in FIG. 14C, but shown in FIG. 15A-D), base 282, port 288 and/or magnetic click connector (271). The protrusion 276 may be sized (e.g., made smaller), shaped (e.g., made tapered) and/or otherwise constructed (e.g., comprising a low friction material and/or coating, including one or more of nylon, PTFE, PEEK, and/or a hydrophilic or hydrophobic coating) in order to reduce the force of engagement with and opening of the valve (not shown). Further alternatively, the protrusion 276 may comprise an external slot or channel, e.g., to facilitate fluid flow around the protrusion and through the open valve. In this case, the protrusion 276 may be made relatively smaller than for example a hollow or annular protrusion, e.g., in order to further reduce the force engagement.

Turning attention to the construction of the valve, representative of which are shown in FIGS. 15A to 15D, the valve may be constructed as different type of valves, such as a flap valve, a spring valve, duckbill valve, an umbrella valve, a diaphragm valve, a dome valve or a slit valve. For example, a flap valve (FIG. 15C, 295; or FIG. 15D, 300) has a flap (297 in FIG. 15C; or FIG. 15D, 302) that covers the hole or opening (FIG. 15A to 15D, 299), and the valve opens when the nose or protrusion of the connector is pushed into the valve. In a particular aspect, the flap may remain closed or shut, in part, due to the relative stiffness of the material of construction, or by a separate elastic member that is over molded, attached or glued to the flap. In another variation, the valve is a spring valve (FIG. 15A, 290). In the spring valve (290), an “I” shaped member (FIG. 15A and FIG. 15B, 292) may be pushed or bias against a plate by an elastic member, such as a spring or an elastic insert. In one variation, the plate comprises one or more holes such that once the “I” shaped member (290) is inserted, it is pushed away by the protrusion, allowing fluids to flow through the hole or opening.

Turning attention to the tubing comprising the balloon catheter 280, in one variation, such tubing may comprise a flexible tubing capable of traversing a bend between 0-90 degrees at bend radius of between 0.5 and 2 cm, i.e., without kinking or obstruction of the catheter lumen 289. Such tubing may be constructed of flexible materials suitable for short or long term implantation such as silicone, polyurethane, TPUs, and/or elastomeric alloys as are know in the art. The tubing may be constructed with a relatively thick wall, e.g., between 0.015-0.050 inches in order to prevent kinking and/or comprise a braided or coiled reinforcing element, as is also known in the art. The length of the line or tubing of the balloon catheter 280 may vary in order to accommodate variation in patient anatomy and/or positioning of the base relative to the patient anatomy while providing sufficient length to reach the target location. Alternatively, the tubing may incorporate one or more pre-shapes, e.g., adapted to known or anticipated patient anatomy such that the tubing can be made to traverse or remain in such anatomy with reduced strain. In one variation, the tubing may incorporate one or more connecting elbows, such as at 90 degrees, 60 degrees, 45 degrees, 15 degrees, 10 degrees or a combination thereof.

In one embodiment where the balloon catheter is used to intermittently drain the bladder, the base 282 may be positioned generally above the belt line of the patient, e.g., in order to enable ease of access by the patient to the base 282, for example when the patient is seated in a wheelchair. In this case, the tubing of the balloon catheter 280 may be tunneled and/or otherwise traverse a path through the external abdominal wall to a point of proximity to the bladder whereupon the tubing may cross through the abdominal wall to enter the bladder.

In certain cases and/or in certain patient positions, the base 282 may be positioned generally above the bladder. For example, in the event that the base is positioned near the umbilicus and the patient is seated, the base 282 may be in an elevated position relative to the bladder. According to this method, the base may be placed above the bladder, i.e., at the umbilicus, for example, and the tubing is tunneled and the device may incorporate or employ a pump or siphon to initiate and/or drain the bladder in spite of the low pressure within the bladder and the elevated position of the base. In another variation, the device provides a tubing emanating from or protruding from the base at a particular angle, such as an obtuse angle, such that the tubing does not require a sharp turning angle to tunnel under the patient's skin.

Unless defined otherwise, all technical terms used herein have the same meanings as commonly understood by one of ordinary skill in the medical arts. Specific methods, devices, and materials are described in this application, but any methods and materials similar or equivalent to those described herein can be used in the practice of the present invention. While embodiments of the invention have been described in some detail and by way of illustrations, such illustrations are for purposes of clarity of understanding only, and are not intended to be limiting. Various terms have been used in the description to convey an understanding of the invention; it will be understood that the meaning of these various terms extends to common linguistic or grammatical variations thereof. Further, while some theoretical considerations may have been advanced in furtherance of providing an understanding of the technology, the appended claims to the invention are not bound by such theory. Moreover, any one or more features of any embodiment of the invention can be combined with any one or more other features of any other embodiment of the invention, without departing from the scope of the invention. Still further, it should be understood that the invention is not limited to the embodiments that have been set forth for purposes of exemplification, but is to be defined only by a fair reading of claims appended to the patent application, including the full range of equivalency to which each element thereof is entitled.

All publications and patent applications mentioned in this specification are herein incorporated by reference to the same extent as if each such individual publication or patent application were specifically and individually indicated to be so incorporated by reference.

What is claimed is:

1. A balloon catheter assembly comprising:
a) a balloon catheter comprising a proximal end, a distal end, wherein the balloon catheter further comprises:
a catheter wall defining a catheter lumen;
an inflatable balloon comprising a proximal end and a distal end, where the inflatable balloon is mounted at the distal end of the catheter and the catheter further comprises a lumen that extends from the balloon to an inflation valve and inflation port;
a base located at the proximal end of the catheter, wherein the base comprises a top end and a bottom end, wherein the base comprises an opening to the catheter lumen, and the base further comprising:
i) a port panel comprising a top end and a bottom end, and a port extending therethrough, wherein the top end of the port panel comprises a magnetic click connector for connecting with a connecting line comprising a corresponding magnetic click connector, and wherein the bottom end of the port panel is attached to the top end of the base; and
ii) an inflation port for inflating the balloon, where the inflation port is mounted on the base and the inflation port further comprises an inflation valve, where the inflation valve is in fluid communication with the balloon using an inflation lumen;
and
b) a connecting line comprising a proximal end and a distal end, wherein the proximal end comprises a first terminal magnetic connector for forming a water tight connection with the magnetic click connector on the port panel.

2. The balloon catheter assembly of claim 1, wherein the magnetic click connector of the top end of the port panel comprises one or more magnetic members mounted on the port panel for securely engaging or connecting the port panel and base to the connecting line.

3. The balloon catheter assembly of claim 1, wherein the first terminal magnetic connector of the connecting line comprises a protrusion for engaging with a valve in the port to form a water tight connection.

4. The balloon catheter assembly of claim 3, further comprising a reversible locking mechanism or locking device for securing the two magnetic click connectors when connected, where the locking mechanism is selected from the group consisting of a twist lock, a locking ring, a male lock lever, a bayonet type twist locking mechanism for quick and easy connect and disconnect.

5. The balloon catheter assembly of claim 1, wherein the base further comprises a one-way valve in the port panel.

6. The balloon catheter assembly of claim 1, further comprising an external pump for pumping fluids through the connecting line, such as for introducing fluids or for removing fluids; or alternatively, a bulb suction device for suctioning fluids.

7. The balloon catheter assembly of claim 1, further comprising a drainage device selected from a drainage bag or a leg bag.

8. A method for draining urine from a bladder using a urinary balloon catheter assembly, the method comprising:
a. installing a properly sized urinary balloon catheter comprising a port panel, wherein the port panel further comprises a magnetic click connector, in a suprapubic tract of a patient;
b. inflating the balloon to securely retain the balloon catheter in the suprapubic tract for an indefinite period of time until a sufficient amount of urine is collected in the bladder;
c. connecting a connecting line comprising a magnetic click connector to the port panel comprising the magnetic click connector by bringing the click connector of the connecting line in close proximity to the click connector of the port panel to allow the two magnetic click connectors to engage and securely connect together to form a water tight seal; and
d. draining the urine from the bladder through the balloon catheter and connecting line using an electrical-mechanical pump, a bulb suction device or by gravity.

9. The method of claim 8, wherein the balloon catheter assembly comprises:
a) a balloon catheter comprising a proximal end, a distal end, wherein the balloon catheter further comprises:
a catheter wall defining a catheter lumen;
an inflatable balloon comprising a proximal end and a distal end, where the inflatable balloon is mounted at the distal end of the catheter and the catheter further comprises a lumen that extends from the balloon to an inflation valve and inflation port;
a base located at the proximal end of the catheter, wherein the base comprises a top end and a bottom end, wherein the base comprises an opening to the catheter lumen, and the base further comprising:
i) a port panel comprising a top end and a bottom end, and a port extending therethrough, wherein the top end of the port panel comprises a magnetic click connector for connecting with a connecting line comprising a corresponding magnetic click connector, and wherein the bottom end of the port panel is attached to the top end of the base; and
ii) an inflation port for inflating the balloon, where the inflation port is mounted on the base and the inflation port further comprises an inflation valve, where the inflation valve is in fluid communication with the balloon using an inflation lumen;
and
b) a connecting line comprising a proximal end and a distal end, wherein the proximal end comprises a first terminal magnetic connector for forming a water tight connection with the magnetic click connector on the port panel.

10. The method of claim 9, wherein the urine is drained into a drainage bag or drained directly into a toilet or disposal container.

11. The method of claim 10, further comprising disconnecting the two magnetic click connector, and plugging the safety plug.

12. A magnetic click connector assembly for connecting a first medical device with a second medical device, a medical device with a connecting line, or a first connecting line with a second connecting line to provide a liquid tight or leak proof magnetic connection, the magnetic click connector assembly comprising:
a) a first magnetic click connector comprising a base further comprising at least one magnetic member; and
b) a second magnetic click connector comprising a base further comprising at least one magnetic member for magnetically coupled with the magnetic member of the first magnetic click connector to form a water tight connection;
wherein the first magnetic click connector is coupled to a medical device or a connecting line and the second magnetic click connector is coupled to a medical device or a connecting line; and
wherein each of the medical device and connecting line comprises a lumen therethrough for the passage of fluid.

13. The magnetic click connector assembly of claim 12, wherein the magnetic member comprises of a structure or shape selected from the group consisting of one or more pellets or beads, one or more rods, one or more circular rings, one or more semi-circular rings, or a combination thereof.

14. The magnetic click connector assembly of claim 13, wherein the magnetic member of the first magnetic click connector comprises of a structure or shape that is the same or different from the second magnetic click connector.

15. The magnetic click connector of claim 12, wherein the magnetic member(s) are mounted on the base and are incorporated into the surface of the bases such that the magnetic member(s) are not exposed to air.

16.-21. (canceled)

22.-55. (canceled)
